(19) United States
(12) Patent Application Publication
(10) Pub. No.: US 2024/0269159 A1
(43) Pub. Date: Aug. 15, 2024

(54) NUTRITIONAL COMPOSITIONS FOR SKELETAL MUSCLE

(71) Applicant: MITOCHOLINE LTD, London (GB)

(72) Inventors: Larisa Andreeva, London (GB); Galina Nonina Skladtchikova, Hellemp (DK)

(21) Appl. No.: 18/566,415

(22) PCT Filed: May 25, 2022
(86) PCT No.: PCT/GB2022/051316

§ 371 Date: Dec. 1, 2023

(30) Foreign Application Priority Data
Jun. 3, 2021 (GB) 2107957.9

(51) Int. Cl. A61K 31/706; A61K 31/14; A61K 31/194; A61K 31/455; A61P 21/00

(52) U.S. Cl. CPC A61K 31/706; A61K 31/14; A61K 31/194; A61K 31/455; A61P 21/00

(57) ABSTRACT

The present invention relates to nutritional compositions for maintaining, restoring and enhancing skeletal muscle energy metabolism. The compositions are useful for improving skeletal muscle tone, strength, and endurance.

### FIELD OF THE INVENTION

The present invention relates to nutritional compositions for maintaining and enhancing skeletal muscle energy metabolism, tone, strength and endurance. The compositions comprise a combination of choline cation and succinate anion (2−).

### BACKGROUND OF THE INVENTION

Achieving peak physical performance has long been a goal for athletic competition and self-improvement. Means for improving physical performance includes prolonged systematic exercise and proper diet. Incorporating good dietary practices, as part of an exercise program can optimize a subject's performance during exercise.

The continual supply of ATP to the fundamental cellular processes that underpin skeletal muscle contraction during exercise is essential for sports performance in events lasting seconds to several hours. Because the muscle stores of ATP are small, metabolic pathways must be activated to maintain the required rates of ATP resynthesis. These pathways include phosphocreatine and muscle glycogen breakdown, thus enabling substrate-level phosphorylation (‘anaerobic’) and oxidative phosphorylation by using reducing equivalents from carbohydrate and fat metabolism (‘aerobic’). The relative contribution of these metabolic pathways is primarily determined by the intensity and duration of exercise. The energy required to support long and intense exercise, e.g. a marathon run, is mainly provided through oxidative phosphorylation in the mitochondria of the active muscles (Spriet L L, 2007, Sports Med 37: 332-336; Hargreves M & Spriet L L 2020 Nature Metabolism 2: 817-828).

Succinate plays a pivotal role in oxidative metabolism in general, and in skeletal muscle specifically. Succinate is a tricarboxylic acid (TCA) cycle intermediate that interacts directly with the mitochondrial electron transport chain (ETC), enabling a ‘shortcut’ route to ATP production via oxidative metabolism.

Recently, it has been shown that dietary succinate (sodium salt of succinic acid) supplementation increased endurance exercise ability in mice. (Wang et al 2019, EMBO Rep 20(9):e47892; Reddy et al., 2020, Cell 183, 1-14). Nicotinamide (NAM), a form of vitamin B3, is a precursor of nicotinamide adenine dinucleotide (NAD), which is a redox-active metabolite. The depletion of NAD has been associated with body tissues ageing and degenerative diseases. It has been reported that NAM administered to adult-onset mitochondrial myopathy patients in the amount of 750-1,000 mg/day increased in all subjects both blood and muscle NAD⁺ levels, muscle strength and mitochondrial biogenesis (clinicaltrials.govNCT03973203; Pirenen E, et al, 2020 Cell Metab., 31(6):1078-1090.e5. doi: 10.1016/j.cmet.2020.04.008).

Formulations of succinate (in a form of di-choline salt) and nicotinamide has been shown to be synergistically effective for increasing the levels of both NAD+ and adenosine triphosphate (ATP) in the brain cells (WO2019002858). Interestingly, these formulations also contain choline. Choline plays a main role in many physiological pathways, including the synthesis of neurotransmitters (acetylcholine). The signaling of the cell membrane (phospholipids) and lipid transport (lipoproteins) among others. Endurance exercise could enhance some of these pathways, increasing the choline demand as a metabolic substrate (Moretti A, et al, 2020, Nutrients 12(7): 2144). According to available research data choline affects skeletal muscle by modulating fat and protein metabolism, inflammation, and autophagy.

Currently available nutritional compositions for consumption before, during and after exercise typically comprise protein lysates or combination of amino acids to restore muscles fiber injuries caused by prolong exercising and mixtures of different carbohydrates to fill the depleted carbohydrate energy stores of the body leading to reduced exercise performance. However, there is still lack of compositions that both enhance oxidative phosphorylation in skeletal muscle, increase autophagy processes, and support physiological adaptation of the muscle mass to a prolonged exercise by supporting the conversion of skeletal muscle fiber from fast twitch to slow twitch induced by endurance or aerobic exercise.

### SUMMARY OF THE INVENTION

A first aspect of the invention relates to a composition comprising, or essentially consisting of, choline and succinate in the molar ratio choline to succinate from about 0.5:3 to about 3:0.5, preferably, about 2:1, for maintaining, enhancing or restoring energy metabolism and/or increasing autophagy and/or supporting physiological adaptation of the skeletal muscle mass to prolong physical exercise. Advantageously, the composition may also comprise nicotinamide (NAM), or a NAM derivate, such as nicotinamide riboside, wherein the molar ratio choline:succinate:NAM/NAM derivate, is about 0.5-3:3-0.5:0.01-10, e.g. about 1:2:0.01-10, preferably, 2:1:0.01-10, such as about 2:1:0.1-1. In some embodiments, the composition may essentially consist of choline cation, succinate anion (2−) and NAM or a NAM derivate in said molar ratio.

A second aspect relates to a composition comprising, preferably essentially consisting of choline and succinate, wherein the molar ratio choline:succinate is from about 0.5:3 to about 3:0.5, e.g. about 1:2, preferably, about 2:1, and, optionally, nicotinamide or a nicotinamide derivate, wherein the molar ratio choline:succinate:NAM/NAM derivate is about 0.5-3:3-0.5:0.01-10, e.g., 2:1:0.01-10, such as about 2:1:0.1-1, for improving physical performance in a subject, requiring an enhanced skeletal muscle strength, muscle tone and endurance.

A third aspect of the invention relates to a composition comprising, preferably essentially consisting of choline, succinate and NAM/NAM derivate, wherein the molar ratio choline:succinate:NAM/NAM derivate is about 0.5-3:3-0.5:0.01-10, e.g., 2:1:0.01-10, such as about 2:1:0.1-1, preferably essentially consisting of choline cation, succinate anion (2−) and NAM or a NAM derivate in said molar ratio, for use in maintaining or enhancing the skeletal muscle energy metabolism in an ageing human, or in a subject having or recovering from a disease.

A third aspect of the invention relates to a composition comprising, or essentially consisting of, choline and succinate in the molar ratio 0.5-3:3-0.5, e.g. between 1:2 to 2:1, preferably, 2:1, and, advantageously, NAM, or a NAM derivate, wherein the molar ratio choline:succinate:NAM/NAM derivate is about 0.5-3:3-0.5:0.01-10, e.g., 2:1:0.01-10, such as about 2:1:0.1-1, preferably essentially consisting of choline cation, succinate anion (2−) and NAM or a NAM derivate in said molar ratio, for use in the dietary management of one or more symptoms and conditions associated with an imbalanced, damaged or reduced skeletal muscle energy metabolism and/or autophagy, such as muscle weakness, muscle pain, low muscle tone, exercise intolerance, lack of muscular endurance and loss of muscle mass.

A fourth aspect of the invention relates to a method for maintaining, improving, or restoring skeletal muscle strength, tone and muscular endurance in a subject, comprising administering to the subject a composition essentially consisting of choline, succinate and, advantageously, NAM or NAM derivate, in a molar ratio from about 0.5-3:3-0.5:0.01-10, e.g., 2:1:0.01-10, such as about 2:1:0.1-1, wherein skeletal muscle of said subject has a weakened or damaged mitochondrial function, or a weakened, unbalanced or damaged autophagy, preferably essentially consisting of choline cation, succinate anion (2−) and NAM or a NAM derivate in said molar ratio.

A fifth aspect, the invention relates to a method for the dietary management of a symptom or condition associated with a musculoskeletal disease or disorder in a subject, e.g. muscle-wasting, muscle degenerative disease, myopathies, age-related decline in muscle function, frailty, pre-frailty, neuromuscular diseases, Duchenne muscular dystrophy, sarcopenia, muscle atrophy and/or cachexia, muscle loss, a muscle function disorder, age-related decline in muscle function, age-related sarcopenia, age-related muscle-wasting, physical fatigue, muscle fatigue, inclusion body myositis, imbalanced, damaged or reduced skeletal muscle energy metabolism in a human, or the dietary prevention of developing, occurring and/or re-occurring a symptom or condition associated with an imbalanced, damaged or reduced skeletal muscle energy metabolism in a human, comprising administering to said human, at least once a day, a composition comprising, choline, succinate in the molar ratio around 2:1, or, advantageously, comprising a composition comprising, choline, succinate and NAM, or a NAM derivate, in the molar ratio from about 0.5-3:3-0.5:0.01-10, e.g., 2:1:0.01-10, such as about 2:1:0.1-1, preferably essentially consisting of choline cation, succinate anion (2−) and NAM or a NAM derivate in said molar ratio.

A sixth aspect of the invention relates to a sport food or beverage for improving physical performance, increasing muscular strength and/or muscular endurance, comprising a composition comprising, or essentially consisting of, choline, succinate in the molar ratio about 0.5-3:3-0.5, e.g. from around 1:2 to 2:1, preferably, around 2:1. Advantageously, a composition may comprise, or essentially consists of, choline, succinate and NAM or a NAM derivate, in a molar ratio about 0.5-3:3-0.5:0.01-10, e.g., about 2:1:0.01-10, such as about 2:1:0.1-1.

In different embodiments, compositions of the invention may be formulated as nutritional compositions, dietary supplements, functional or medical foods/beverages. The compositions are for the oral administration and may be administered in one or more doses daily for a period of one or more days. A single oral dose may contain from around 10 to around 5000 mg of a composition of the invention.

Compositions of the invention may further comprise additional ingredients, such as e.g. vitamins B9, B6, B12, or minerals such as Ca or Mg, or other ingredients, that are beneficial for maintaining and/or restoring the skeletal muscle healthy functioning and/or improving its performance.

Subject who would benefit from the compositions of the invention may be any human subject, including any healthy human subject with or without particular demand for improving his/her own physical performance, ageing individuals and/or individuals who are weakened due to a disease or certain mental condition, such as e.g. psychological or environmental stress, fatigue, insomnia or mental depression.

Preferably, choline and succinate in compositions of the invention are derived from dicholine succinate salt CAS No: 109438-15-5 (interchangeably, identified herein as choline succinate (2:1) salt or di-choline succinate or DISU).

### DESCRIPTION OF THE FIGURES

FIG. 1 shows representative results of treatments of cultures of primary myotubes either with two different blends of DISU and NAM (blend 1: DISU 100 μM+NAM 40 μM; blend 2: DISU 50 μM+20 μM NAM; the molar ration choline:succinate:NAM in both blends is 2:1:0.4), or with single compounds of the blend, i.e. choline (100 μM), succinate (50 μM), NAM (50 μM):
1A—evaluation of autofluorescence of NADH;
1B—evaluation of the mitochondrial redox index (corresponds to NADH-dependent mitochondrial respiration=;
1C—evaluation of the mitochondrial pool of NADH (measured as a Δ between FCCP and NaCN NADH autofluorescence).

FIG. 2 shows representative results of the evaluation of autophagy in primary cultures of myotubes treated with a blend of DISU and NAM (50 UM DISU and 20 μM NAM; the molar ratio choline:succinate:nicotinamide is 2:1:0.4) evaluated as the level of co-localization of mitochondria and lysosomes. The upper panel: confocal images of cells loaded with two dyes MitoTracker Green (labelling mitochondria) and LysoTracker Red (labelling lysosomes); the lower panel: quotative evaluation of co-localisation of mitochondria and lysosomes in cells. The cells were incubated in the absence (control, panel A) or in the presence of 50 μM DISU and 20 UM NAM (treated cells, panel B). Scale bars in images 20 μm. Values are presented as mean±SD. **P<0.001. Independent experiments (n) are indicated as dots in the scatter plot.

FIG. 3 shows representative results of the evaluation of treatment of cultures of primary myotubes with a blend of DISU and NAM (50 μM DISU+20 μM NAM; the molar ratio choline:succinate:NAM is 2:1:0.4): A—measurement of autofluorescence of FAD+++; B—evaluation of the mitochondrial pool of FAD++.

FIG. 4 shows representative results of the evaluation of energetic capability of primary myotubes stimulated to contract with caffeine and either treated or non-treated (control) with a blend of 50 μM DISU and 20 μM NAM (the molar ratio choline:succinate:NAM is 2:1:0.4). The myotube energetic capability is evaluated as duration of the caffeine stimulated myotube contraction in the presence or absence of DISU+NAM (i.e. time to collapse of myotube contraction).

### DETAILED DESCRIPTION OF THE INVENTION

Support of skeletal muscle energy metabolism is of crucial importance both during intense physical activity and in case of muscle cell function/muscle mass decline associated with ageing or disease. Well-functioning skeletal muscle energy metabolism is important for sustained and tonic contractile events, maintenance of energy homeostasis, and the alleviation of fatigue. There is a need for nutritional compositions capable of maintaining and increasing energy levels for an extended period of time, supporting muscle cells metabolome exhausted due to intensive training or due to disease or ageing, and maintaining and enhancing the skeletal muscle fiber strength and endurance, and adaptability to prolonged exercise.

The present invention relates to synthetic compositions that are safe and effective to maintain, enhance and restore skeletal muscle energy metabolism in mammals in general, and in particular, in human subjects. The compositions can advantageously be used alone or in a combination with any diet and/or therapy to support or re-establish a proper skeletal muscle energy metabolism and to enhance the skeletal muscle strength and endurance in human subjects, including both physically active healthy human individuals and physically weakened individuals that are suffering or recovering from a disease of any age and in a combination with any diet and/or therapy.

Accordingly, the invention in general relates to compositions that are effective to maintain, enhance and restore skeletal muscle energy metabolism, and maintain, enhance and restore physical performance in human subjects. Compositions of the invention comprise at least two essential compounds, choline (cation) and succinate (anion 2−) in a molar ratio choline:succinate ranging from around 0.5:3 to around 3:0.5, such as from around 1:2 to around 2:1, preferably, around 2:1. In some embodiments, the compositions additionally may comprise at least three compounds which are choline (cation) and succinate (anion 2−) and nicotinamide (NAM), or a NAM analog, e.g nicotinamide riboside, wherein the molar ratio choline:succinate:NAM/NAM analog is around 0.5-3:3-0.5:0.01-10, preferably, around 2-1:1-2:0.01-10, e.g. 2:1:0.1-1. It is surprisingly found that choline (cation) and succinate (anion 2−) in the indicated molar ratio, advantageously, in a combination with NAM, preferably, in the molar ratio choline:succinate:NAM around 2-1:1-2:0.01-10, e.g. 2:1:0.1-1, can synergistically enhance mitochondrial function of skeletal muscle cells and boost the production of “energy” molecules ATP (Adenosine 5′-triphosphate), NAD (Nicotinamide Adenine Dinucleotide) and FAD (Flavin Adenine Dinucleotide).

ATP is the main source of energy for all cellular processes. NAD and FAD are essential intracellular coenzymes that play key roles in cellular oxidation-reduction (redox) reactions and are responsible for accepting high-energy electrons and carrying them to the electron transport chain to synthesize ATP. The present inventors surprisingly found that a combination of choline and succinate, where the molar ration of choline:succinate is around 1-2:2-1, preferably 2:1, strongly enhances the production of NAD, FAD and ATP in mitochondria of muscle cells. The effect is even stronger, when such composition further comprises NAM, and the molar ratio choline:succinate:NAM is around 1-2:2-1:0.01-10, preferably 2:1:0.1-1. Preferably, choline and succinate of the composition are derived from di-choline succinate salt (DISU), however, other salts of choline and succinic acid may be used as well. None of components of the three-compound composition alone, nor in a combination of any two of the tree compounds were able to increase the production of said energy molecules to the same level as when both DISU and NAM were present, still, a less profound effect of a combination of choline and succinate in the molar ratio choline:succinate from around 0.5:3 to around 3:0.5, such as from around 1:2 to around 2:1, preferably, around 2:1, was observed as well. It is also found that oral administration of a composition essentially consisting of DISU and NAM to human individuals for a period of time of one or more days leads to at least one of the following: an increased skeletal muscle strength, tone and endurance.

All terms and definitions explained throughout the specification of the invention relate to all aspects and embodiments of the invention, unless otherwise specified.

The wording “composition essentially consisting of” <named compounds> means that the named compounds of the composition are major compounds of the composition and contributing to the biological effect(s) associated with the composition.

The term “synthetic” in the present context means a man-made composition. The synthetic compositions of the invention may comprise both synthetically prepared molecules that are structurally identical to the molecules that naturally occurring in the living bodies, and artificial molecules that do not have natural structural equivalents.

The terms “about” and “around” mean a deviation from the indicated value by 0.01% to 10%, such as from 0.5% to 5%.

The term “choline cation” (or “choline”) means the cation having the chemical formula C₅H₁₄NO⁺ (CAS No. 62-49-7).

The term “succinate anion” (or “succinate” or “succinate (2−)” means in the present context succinic acid anion (2−) having the chemical formula C₄H₄O₄⁻² (CAS No. 110-15-6), The terms “di-choline succinate”, “choline succinate salt (2:1)” and “DISU” are interchangeable and mean the molecule of formula (1):

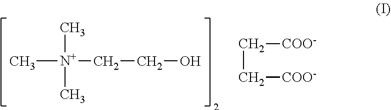

The term “nicotinamide” or “NAM” means the molecule identified with CAS No. 98-92-0. The term “derivate of nicotine amide” of NAM derivate” means a molecule that is derived from NAM by a synthetic process, i.e. NAM is a start molecule for the synthesis of the derivate such as nicotinamide riboside (CAS No: 1341-23-7) or nicotinamide mononucleotide (CAS No: 1094-61-7).

The invention relates to compositions comprising, (in some embodiments, essentially consisting of) choline cation and succinate anion (2−); preferably the choline cation and succinate anion are present in the molar ratio about 0.5-3:3-0.5, preferably 2:1, preferably, said cation and anion are present in form of choline succinate salt (2:1) (DISU), i.e. DISU is a part of the composition. Alternatively, the choline cation of the composition may derive from another salt of choline, e.g. choline bitartrate (CAS No. 87-67-2), and succinate anion may derive from another salt of succinic acid, e.g. succinic acid disodium salt (CAS No. 6106-21-4). Compositions essentially consisting of choline bitartrate, choline butyrate, choline chloride or choline fumarate, and succinic acid di-sodium or di-ammonium salt may be preferred in some embodiments of the invention. The molar ratio of choline cation and succinate anion (2−) in such compositions is about 2:1.

Advantageously, a composition of the invention comprising choline and succinate (molar ratio about 0.5-3:3-0.5, preferably, 2:1) comprises NAM or a NAM derivate. The amount of NAM, or a NAM derivate, in compositions of the invention may vary within the range of molar ratio of choline to succinate to NAM/NAM derivate, e.g. the molar ratio may be within the range 0.5-3:3-0.5:00.1-10, such as from about 2:1:0.01 to about 2:1:10. Compositions with a molar ratio of the individual compounds within this range act synergistically in enhancing mitochondrial function in skeletal muscle cells reflected by significant increase in the mitochondrial production of major energy molecules NAD, FAD and ATP. These compositions are effective in dietary management of symptoms and conditions associated with imbalanced or weakened energy metabolism, weakened or damaged mitochondrial function and/or weakened or damaged autophagy in skeletal muscle cells, such as muscle weakness, muscle pain, low muscle tone, exercise intolerance, lack of muscular endurance and loss of muscle mass. Furthermore, the compositions are beneficial as nutritional food supplements to improve physical performance of both healthy physically active human subjects and human subjects that are physically weaken due to ageing, disease or psychological or environmental stress.

To obtain the above effects, the essential compounds of the composition, i.e. choline, succinate, and, advantageously, NAM, or a NAM derivate, are to be present in the compositions in an so-called “effective amount”. The effective amount of the compounds may vary depending on the aim and/or method of use, and/or subject in need. These embodiments are discussed below and exemplified by non-limiting working examples.

In some embodiments, a composition of the invention comprising choline cation, succinate anion (2−) and NAM may further comprise creatine (CAS No. 57-00-1), or a creatine precursor, such as e.g. amino acids glycine and arginine. Other useful additives to a composition comprising choline cation, succinate anion (2−) and NAM, or a NAM derivate, are discussed below.

Compositions of the invention can be advantageously used for maintaining, enhancing and/or restoring a proper level of skeletal muscle energy metabolism in a human, wherein the term “proper level of skeletal muscle energy metabolism” means a dynamic capability of the skeletal muscle cells to generate energy by producing biological “energy” molecules NAD, FAD and ATP in the amounts that are sufficient to maintain optimal skeletal muscle functioning under normal conditions, including the muscle tone, strength of the contraction and the contraction endurance, and in the amounts that can compensate for an increased energy consumption under extraordinary conditions, e.g. such as an intense and/or prolonged physical exercise. The term “imbalanced, damaged or reduced skeletal muscle energy metabolism” is synonymous with the term “improper level of skeletal muscle energy metabolism”, and the present content means that a dynamic capability of the skeletal muscle cells to generate energy by producing biological “energy” molecules NAD, FAD and ATP in the amounts that are sufficient to maintain optimal skeletal muscle functioning under normal conditions is weakened, e.g. due to a disease, ageing, physical trauma, environmental or psychological stress, etc. The proper level of skeletal muscle energy metabolism may differ from one human individual to another, and may depend on age, and/or physical capability of the individual. The proper level may be defined according to existing standards for physical performance, e.g. using physical performance studies described in Examples 3 and 4 of the specification.

The wording “maintaining skeletal muscle energy metabolism” means in the present context that compositions of the invention provide support for the generation of energy molecules ATP, NAD and FAD in mitochondria to keep the energy metabolism in skeletal muscle of an individual on a proper level at any time. The wording “restoring skeletal muscle energy metabolism” means in the present context that compositions of the invention provide an extraordinary support in generation of energy molecules ATP, NAD and FAD by mitochondria of an energy-depleted and weakened skeletal muscle of an individual due to, e.g., a disease, ageing, physical trauma, environmental or psychological stress factor etc. experienced or is experiencing by said individual, and helping thereby to said individual to overcome the energy exhaustion and restoration of the physical condition of the individual's skeletal muscle to a proper level. The wording “enhancing skeletal muscle energy metabolism” means in the present context that compositions of the invention, by supporting generation of energy molecules of ATP, NAD and FAD in mitochondria of skeletal muscle of a healthy individual, increase the energetic potential of skeletal muscle of said individual thereby allowing the individual's ability to efficiently use skeletal muscles over a longer period and under occasional stressful conditions, like an acute disease, physical trauma, psychological stress, etc. Advantageously, for the maintaining, enhancing and/or restoring a proper level of the skeletal muscle energy metabolism, the compositions can be used by human individuals, both by healthy individuals and medical patients, as an everyday dietary supplement, as it is safe to use in a combination with any diet and therapeutic treatment. Advantageously, in one embodiment, the compositions may be used for maintaining or, preferably, enhancing the skeletal muscle energy metabolism in an ageing human. The term “ageing human” in the present context generally relates to a human individual over 24-25 years old, preferably over 34-35 years old, such as 30 to 45, around 50 or 60 years old or older. In one embodiment, the composition can be advantageously used for the prophylaxis of ageing of skeletal muscle. It is well known that skeletal muscle function declines and the muscle mass diminishes with ageing. Used on everyday basis for maintaining a proper skeletal muscle energy metabolism by a relatively young human individual, e.g. of 20-40 years old, compositions of the invention have an advantageous capability to maintain the muscle mass and muscle strength and endurance at healthy levels through ageing and enhance physical performance of said individuals for longer compare to individuals of the same aged who did not intake the same compositions. Thus, compositions of the invention helps to decreasing the speed of ageing associated with the depletion of skeletal muscle mass and decline of skeletal muscle function.

As already mentioned above, human subjects according to invention may be healthy physically active individuals, individuals suffering of or recovering from a disease, or individuals whose skeletomuscular activity is affected by a physical injury or psychological or environmental factors.

By “physical activity is meant any bodily movement produced by skeletal muscles that require energy expenditure. By “healthy physically active individual” in the present context is meant a human subject who has a good physical health, i.e. an individual that has his/her bodily functions and processes working normally, not necessarily at their peak, but not significantly deviating from their peak to affect individual complete physical, mental, and social well-being, in particular bodily functions and processes relating to musculoskeletal activity. In one preferred embodiment, a healthy physically active individual is a human subject who has regular periods of intensive physical activity requiring significant energy expenditure, e.g. a subject who is doing physical exercise to improve his/her physical performance such as a sportsman. Compositions of the invention can help to such individuals to restore, maintain and/or enhance the skeletal muscle energy metabolism and thereby to restore, maintain and/or enhance muscular tone, strength and endurance.

In another preferred embodiment, a healthy human subject is an ageing human individual. The energy production and mitochondrial function decline in ageing skeletal muscle cells, consequently skeletal muscle tone, strength and endurance decrease with age. The compositions of the invention can be a beneficial supplement to the diet of ageing human individuals, helping to their musculoskeletal system to overcome the age-related muscle function decline and thereby delay the onset of skeletal muscle ageing. The compositions of the invention are, surprisingly, effective in enhancing autophagy in skeletal muscle cells, which is essential for maintaining the muscle mass and healthy functioning skeletal muscle tissue. Therefore, compositions of the invention may be useful for restoring, maintaining and/or enhancing skeletal muscle cell autophagy. Autophagy declines during muscle aging. Boosting basal autophagy of cells by promoting the selective degradation of misfolded proteins and dysfunctional organelles would protect the skeletal muscle from dysfunction induced and accelerated by ageing.

Compositions of the invention may also be advantageously used by individuals suffering of or recovering from a disease, or individuals affected by a physical injury or environmental or psychological stress factors. Often such individuals have a decreased level of energy production in all body cells, including skeletal muscle, and, in general, weakened skeletomuscular function and decreased physical activity. Accordingly, in one preferred embodiment, the invention relates to a human subject who is suffering of or recovering from a disease, or to an individual affected by a physical injury or an environmental or psychological stress factor, such as psychological pressure, fatigue, insomnia, mental depression, seasonal affective disorder (SAD), etc. The compositions of the invention will boost energy production in skeletal muscle cells of these individuals and increase muscle tone, strength and endurance, helping thereby to these individuals to overcome their physical incapability.

In one embodiment, the invention relates to human subjects who are suffering of or recovering from a musculoskeletal disease, such as e.g. muscle degenerative diseases, myopathies, age-related decline in muscle function, frailty, pre-frailty, neuromuscular diseases, Duchenne muscular dystrophy, sarcopenia, muscle atrophy and/or cachexia, muscle loss, a muscle function disorder, age-related sarcopenia, age-related muscle-wasting, physical fatigue, muscle fatigue, inclusion body myositis, sporadic inclusion body myositis. In one embodiment, a human subject is an individual who is diagnosed with a metabolic myopathy, such as acid maltase deficiency (AMD, Pompe disease, glycogenosis type 2, lysosomal storage disease), carnitine deficiency, carnitine palmityl transferase deficiency (CPT deficiency), debrancher enzyme deficiency (Cori or Forbes disease, glycogenosis type 3), lactate dehydrogenase deficiency (glycogenosis type 11), myoadenylate deaminase deficiency, phosphofructokinase deficiency (Tarui disease, glycogenosis type 7), phosphogylcerate kinase deficiency (glycogenosis type 9), phosphogylcerate mutase deficiency (glycogenosis type 10), phosphorylase deficiency (McArdle disease, myophosphorylase deficiency, glycogenosis type 5). These diseases may be completely or partly overcome by adjusting diet to draw energy more efficiently from unaffected pathways. Compositions of the invention could be a beneficial diet supplement contributing to recovery these diseases by adjusting, bypassing and/or enhancing energy channels in skeletal muscle cells of the affected human subjects.

Compositions of the invention may be formulated as nutraceutical, nutritional or pharmaceutical compositions. These formulations will comprise effective amounts of the essential ingredients of the composition of the invention in an appropriate molar ratio, as described above. The different formulations can be prepared according to standard rules and proceeding established in the corresponding art.

The term “nutraceutical” means a pharmaceutical-grade standardized nutrient. The term “pharmaceutical” in the present content means a pharmaceutical grade compound prescribed as medicament to treat a disease. The term “nutrient” means in the present context substance that provides nourishment essential for the maintenance of life of a human. The term “nutritional” in the present context means that the composition is for the dietary supplementation of a human individual. The term “dietary supplement” means a product taken by mouth that contains a dietary ingredient, e.g. a nutrient, intended to supplement the diet.

The amounts of choline cation, succinate anion (2−) and NAM, e.g. DISU and NAM, in a composition of the invention may be adjusted for use by a particular individual or a group of individuals according to the individual's needs, age, physiological conditions, etc., and depending on the dosage form and administration regime For example, the amount of NAM in the composition may vary from 4 mg to 4000 mg per serving, served as one or more dosages a day, such as about 20-2000 mg per serving, served as one or several dosages per day, or about 10-1000 mg per serving served as one or several dosages per day, etc, wherein the daily dose of NAM will depend on the dietary demand of a concrete human individual or a group of human individuals. Non-limiting working examples of dietary compositions are described in Examples. A daily intake of up to 4000 mg of NAM in the composition is considered safe and effective for any described herein purpose. The amount of DISU per serving may vary from 10 mg to 1000 mg per serving, and it can be served in one or more dosages a day. According to the invention, an individual may intake a composition comprising up to 4000 mg NAM, or a NAM derivate, and up to 1000 mg DISU, or the corresponding amounts of choline cation and succinate (2−) anion derived from other salts of choline and succinic acid, daily without having any side effects.

In one preferred embodiment, a composition of the invention is a nutritional composition and comprises essentially DISU and NAM, wherein the molar ratio of choline cation, succinate anion (2−) and nicotinamide in the composition is about 2:1:0.1-1, such as about 2:1.:0.6-0.9 or about 2:1:0.2-0.5, e.g. about 2:1:0.4, correspondingly. The term “about” in the present context means a 1-10% deviation from the indicated value, e.g. the molar choline:succinate ratio indicated as 2:1 includes the molar ratios 1.7:0.9 or 2.3:1.1.

Preferably, intake of compositions of the invention is continuous for a period of more than one day, preferably at least 5-7 days (one week) or, preferably, for a longer period, such as from 10-14 days to 20-30 days, or, preferably, longer, e.g. 2-3 months, 3-6 months, 12 months or longer. However, according to the invention, a single intake of a composition of the invention could also be beneficial for the skeletomuscular function of individuals, especially, if the individual is a sportsmen, and the composition is consumed right after said sportsmen' intense workout. There is no limitation for how long the composition can be administered as a dietary supplement. In general, the longer intake of the composition leads to more pronounced beneficial effects. The intake can be interrupted at any time and resumed again when the individual feels that it is needed, e.g. in connection with changes in individual's life style, health, individual's physical/mental conditions, or age. A dietary manager of ordinary skill can readily determine the amounts of ingredients of a dietary composition of the invention according to the accepted rules and regulations.

As discussed above, dietary compositions of the invention are useful for restoring, maintaining and/or enhancing muscular tone, strength and endurance in healthy and weakened human individuals. “Muscular tone” is defined herein as the tension in a muscle at rest. It is the muscle's response to an outside force, such as a stretch or change in direction. Appropriate muscle tone enables our bodies to quickly respond to a stretch. The term “muscular strength” means in general how much force a human individual can exert on a or how much weight a human individual can lift at once. The term “muscular endurance” refers to the ability of a muscle to sustain repeated contractions against resistance for an extended period of time.

Accordingly, use of a combination of choline cation, succinate anion (2−) and nicotinamide, or a nicotinamide derivate, as described herein, in a human, for
restoring, maintaining and/or enhancing skeletal muscle energy metabolism;
restoring, maintaining and/or enhancing muscular strength and endurance;
maintaining and/or enhancing skeletal muscle function;
treating a condition or a symptom associated with an imbalanced, damaged or reduced skeletal muscle energy metabolism, or reducing the risk of development or re-occurrence thereof;
treating physical impairment associated with an imbalanced, damaged or reduced skeletal muscle energy metabolism, or reducing the risk of or delaying the onset thereof, in said human is one of the preferred embodiments of the invention. Compositions comprising different combinations of choline cation, succinate anion (2−) and nicotinamide, or a nicotine derivate, as described herein, are beneficial and safe, and can be combined with a broad spectrum other nutritional compounds in beverages, nutritional supplements and foods or used alone without further additional ingredients.

The invention also includes aspects relating to methods of dietary management of symptoms and conditions associated with imbalanced, damaged or reduced skeletal muscle energy metabolism, loss of balance due to muscle weakness and unstable joints, weakened muscle tone, strength and endurance, said methods comprising a step of administration of a composition according to the invention to a human individual in need. The term “dietary management” in the present context means the practice of providing a nutritional option for individuals and groups with health concerns instead of a therapeutic intervention or as a prophylactic treatment, preferably under supervision of a dietary or medical professional. Advantageously, the compositions of the invention can be used for in dietary prophylaxis of primary and/or secondary development or occurrence of a symptom or condition associated with an imbalanced, damaged or reduced skeletal muscle energy metabolism in a human, including reducing the risk of occurrence and re-occurrence of the same symptom or condition, and reducing the strength and duration of the symptom or condition that is occurred for the first time or is re-occurring. The term “primary” means that the symptom or condition occurs in the human for the first time; the term “secondary” means that the symptom or condition is reoccurring. Accordingly, a method for the dietary prevention of development, occurrence and/or re-occurrence of a symptom or condition associated with an imbalanced, damaged or reduced skeletal muscle energy metabolism in a human, wherein said symptom or condition may be any of the described herein, is one of the aspects of the invention. The term “prevention” in the present context means mitigation of the risk or likelihood of occurrence and/or re-occurrence of a symptom or condition associated with an imbalanced, damaged or reduced skeletal muscle energy metabolism. According to the invention, compositions comprising the essential ingredients described herein, i.e. choline, succinate and NAM, in the molar ratio of around 0.5-3:3-0.5:0.01-10, e.g. about 1:2:0.01-10, such as 2:1:0.01-10, preferably about 2:1:0.1-1, administered orally to healthy individuals on a daily basis during at least 5-7 days, preferably for longer periods of time, are capable of creating a long-lasing increase in the energetic and renewal potential of the skeletal muscle cells allowing thereby the skeletal muscle cells functioning well and not deteriorate for longer. Non-limiting examples of symptoms or conditions that could be advantageously relieved by the composition of the invention are symptoms or conditions associated with musculoskeletal diseases and disorders, muscle-wasting, muscle degenerative disease, myopathies, age-related decline in muscle function, frailty, pre-frailty, neuromuscular diseases, Duchenne muscular dystrophy, sarcopenia, muscle atrophy and/or cachexia, muscle loss, a muscle function disorder, age-related sarcopenia, age-related muscle-wasting, physical fatigue, muscle fatigue, inclusion body myositis, sporadic inclusion body myositis or one or more metabolic myopathies described above.

The term wording “imbalanced, damaged or reduced” in the present context means that an individual's skeletal muscle energy metabolism is not on a proper level (as discussed above), but weakened by a disease, physiological, psychological or environmental condition, or ageing of the individual.

As mentioned above, in some embodiments the invention relates to nutritional compositions comprising, besides the essential compounds of the invention (as discussed above), additional nutrients.

Examples of suitable additional ingredients include, but are not limited to, carriers, minerals, carbohydrates, lipids, vitamins, co-factors, buffers, flavors and sweeteners, inorganic salts, cations and anions typically abandoned in natural drinking water, taste modifying and/or masking agents, carbon dioxide, amino acids, organic acids, antioxidants, preservatives, and colorants.

The nutritional compositions can be combined with one or more carriers and used in the form of ingestible tablets, buccal tablets, troches, capsules, elixirs, suspensions, syrups, wafers, chewing gums, foods, beverages, and the like. Non-exclusive examples of ingredients which can serve as carriers include water; sugars, such as glucose, lactose, and sucrose; cellulose, and its derivatives; starches, such as corn starch and potato starch; powdered tragacanth; malt; gelatin; talc; excipients, such as cocoa butter; oils, such as olive oil, peanut oil, cottonseed oil, corn oil and soybean oil; glycols, such as propylene glycol; esters, such as ethyl oleate and ethyl laurate; polyols, such as glycerin, mannitol, sorbitol, and polyethylene glycol; agar; buffering agents; water; pH buffered solutions; and other non-toxic compatible substances employed in formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions. Non-exclusive examples of antioxidants are Vitamin E, ascorbic acid, carotenoids, aminoindoles, Vitamin A, uric acid, flavonoids, polyphenols, herbal antioxidants, melatonin, lipoic acids, and mixtures thereof.

Compositions of the invention may additionally comprise useful inorganic salts, cations and/or anions. Non-exclusive examples of useful inorganic salts are sodium carbonate, sodium bicarbonate, potassium chloride, magnesium chloride, calcium chloride, and mixtures thereof. Non-exclusive examples of useful cations are sodium, potassium, magnesium, calcium, zinc, iron, and mixtures thereof. Non-exclusive examples of useful anions are fluoride, chloride, bromide, iodide, carbonate, bicarbonate, sulfate, phosphate, and mixtures thereof.

The pH of the liquid compositions could be adjusted to neutral using different buffers. Non-exclusive examples of suitable buffers are phosphate buffer, glycine buffer, citrate buffer, acetate buffer, carbonate buffer, tris-buffer, triethanolamine buffer, and succinate buffer.

Compositions may comprise some flavoring compounds, sweeteners and/or colorants. Non-exclusive examples of suitable flavors are synthetic flavor oils; flavoring aromatics and naturals oils such as cinnamon oil, oil of wintergreen, peppermint oils, clove oil, bay oil, anise oil, eucalyptus, thyme oil, cedar leave oil, oil of nutmeg, oil of sage, oils of citrus fruits, oil of bitter almonds, and cassia oil; plant extracts, flowers, leaves, fruits, vanilla, chocolate, mocha, coffee, apple, pear, peach, citrus such as lemon, orange, grape, lime, and grapefruit; mango, strawberry, raspberry, cherry, plum, pineapple, and apricot, and combinations thereof. Non-exclusive examples of suitable sweeteners are natural and synthetic sweeteners. Non-exclusive examples of natural sweeteners are naturally occurring substances, sucrose, extracts from naturally occurring substances; extracts of the plant Stevia rebaudiana Compositae Bertoni such as stevia, steviol, rebaudiosides A-F, and dulcosides A and B; extracts of Thladiantha grosvenorii such as mogroside V and related glycosides and triterpene glycosides; phyllodulcin and its derivatives; thaumatin and its derivatives; mogrosides such as mogroside IV, mogroside V, siamenoside, and mixtures thereof; genus Siraitia including S. grosvenorii, S. siamensis, S. silomaradjae, S. sikkimensis, S. Africana, S. borneesis, and S. taiwaniana; naturally-occurring glycosides; and active compounds of plant origin having sweetening properties, and mixtures thereof. Non-exclusive examples of synthetic sweeteners are aspartame saccharin, and mixtures thereof. Non-exclusive examples of suitable colorants are dyes suitable for food such as those known as FD&C dyes, natural coloring agents such as grape skin extract, beet red powder, titanium dioxide, and beta-carotene, annatto, carmine, chlorophyll, paprika, and mixtures thereof.

In some embodiments, the compositions may include useful organic acids and/or amino acids. Non-exclusive examples of useful organic acids are acetic acid, butyric acid, malic acid, pyruvic acid, glutamic acid, citric acid, omega-3 unsaturated acids, linoleic acid, linolenic acid, eicosapentaenoic acid, docosahexaenoic acid, aspartic acid, and mixtures thereof. Non-exclusive examples of useful amino acids are Glycine, Arginine, L-Tryptophan, L-Lysine, Methionine, Threonine, Levocarnitine, and L-carnitine.

In some embodiments, the compositions may additionally comprise vitamins and/or cofactors. Non-exclusive examples of useful vitamins are thiamin, riboflavin, panthothenic acid, biotin, folic acid, pyridoxine, vitamin B12, lipoic acid, vitamin A, vitamin D, vitamin E, ascorbic acid, choline, carnitine; alpha, beta, and gamma carotenes; vitamin K, and mixtures thereof. Non-exclusive examples of useful co-factors are thiamine pyrophosphates, flavin mononucleotide, pyridoxal phosphate, biotin, tetrahydrofolic acid, Coenzyme A, coenzyme B12, 11-cis-retinal, 1,25-dihydroxycholecalciferol and mixtures thereof.

In one embodiment a nutritional composition of the invention may comprise compounds that are able to increase the blood circulation, e.g. an extract of Ginkgo biloba or ginseng. In some embodiments, a composition of the invention may comprise an anti-oxidant, e.g. astaxanthin, resveratrol, flavonoids.

The above described and other beneficial optional ingredients generally are used at levels from about 0.0005% to about 10.0% by weight of a composition consisting of the essential components.

Such nutritional compositions of the invention may be formulated as any type of nutritional product such as a food, a beverage, a dietary supplement, a functional food, and a medical food. In one preferred embodiment, a composition of the invention is an aqueous nutritional composition, e.g. a drink or beverage, such as a sport beverage. Preferred embodiments of formulation of the compositions of invention include sport nutritional supplements, sport foods and sport beverages. In one preferred embodiment a sport nutritional supplement, food or beverage comprises from around 10 mg to around 5000 mg of a composition essentially consisting of choline and succinate in the molar ratio of about 0.5-3:3-0.5, e.g. 1:2, preferably, 2:1. In another embodiment, a sport nutritional supplement, food or beverage comprises from around 10 mg to around 5000 mg of a composition essentially consisting of choline, succinate and nicotinamide, or a nicotinamide derivate, in the molar ratio, preferably, from around 2:1:0.01 to around 2:1:1. The other suitable molar ratios of the compounds are discussed throughout the specification. Preferably, choline and succinate are present in a sport nutritional supplement, food or beverage of the invention the form of DISU. A sport nutritional supplement, food or beverage of the invention is useful for maintaining, improving or restoring physical performance, muscular strength and/or muscular endurance in a human subject who is spending time on physical exercise in order to improve his/her physical performance and increase the muscle mass.

In one embodiment, a composition comprising from around 10 mg to around 5000 mg and essentially consisting of choline and succinate in the molar ratio about 2:1, or a composition comprising from around 10 mg to around 5000 mg and consisting of choline, succinate and nicotinamide, or a nicotinamide derivate, in the molar ratio from around 2:1:0.01 to around 2:1:10 may be advantageously used as a supplement to a ketogenic diet or the composition may be included in a ketogenic drink or food product. Elevated circulating ketone bodies from ketogenic diet used by skeletal muscle as a fuel alter substrate competition for respiration, while improving oxidative energy transduction under certain conditions, such as endurance exercise. Consequently, combination of compositions of the invention with nutritional ketosis may help to unlock greater human metabolic potential, e.g. in endurance exercise.

In practicing the invention, the essential compounds of compositions of the invention can be prepared by any process known in the art or obtained from a commercial manufacturer, e.g. nicotinamide or its derivatives, choline bitartrate, succinate disodium salt, may be obtained from Merck. DISU can be prepared by the reaction of choline hydroxide (CAS No. 123-41-1) with succinic acid (CAS No. 110-15-6) as, e.g. described in WO2009/022933A1. The nutritional compositions comprising the essential and, optionally, additional ingredients described herein, may be prepared by procedures well-known from the art.

In one embodiment, a nutritional composition of the invention can be used as a component of a food product. Nonexclusive examples of food products include regular foods, dietary supplements, beverages, and medical foods. The term “medical food” refers to a food which is formulated to be consumed or administered enterally under the supervision of a physician and which is intended for the specific dietary management of a disease, condition, or disorder.

Preferably, the nutritional compositions are formulated for oral administration and preferably administered to a human orally for a period of one or more days, preferably for at least 5-7 days or, even more preferably, for a longer period of time longer (as discussed above). However, human subjects will also benefit from a single oral administration of a composition of the invention, e.g. human individuals who would intake the composition right after their physical workout. The compositions of invention would help such individuals to more rapidly compensate for exhaustion of energy in their skeletal muscle by boosting the production of energy molecules in the skeletal muscle cells.

Non-limiting embodiments of compositions of the invention and their applications are summarized below:
A composition comprising choline and succinate in the molar ratio about 2:1, for maintaining, enhancing and/or restoring skeletal muscle energy metabolism, and/or autophagy in skeletal muscle in a subject; preferably, the composition comprise, or essentially consists of choline, succinate and nicotinamide, or a nicotinamide derivate, in the molar ratio from about 2:1:0.01 to about 2:1:10. Preferably, the composition comprising choline succinate (2:1) salt. The nicotinamide derivate is preferably nicotinamide riboside. The amount of nicotinamide, or the nicotinamide derivate, is from around 10 mg to around 4000 mg, and the amount of choline succinate (2:1) salt is, preferably, from around 10 mg to around 1000 mg.
A composition comprising choline and succinate in the molar ratio about 2:1, for use in maintaining, improving, or restoring skeletal muscle tone strength and endurance in a subject. The composition comprises, preferably, essentially consists of choline, succinate and nicotinamide, or a nicotinamide derivate, in the molar ratio from about 2:1:0.01 to about 2:1:10. The composition preferably comprises choline succinate (2:1) salt. The nicotinamide derivate is preferably nicotinamide riboside. The amount of nicotinamide, or the nicotinamide derivate, is from around 10 mg to around 4000 mg. The amount of choline succinate (2:1) salt is from around 10 mg to around 1000 mg. The composition is preferably a nutritional supplement, e.g. for an ageing human subject, a physically active human subject. The composition is formulated as, e.g., a sport food, a sport beverage or a sport supplement. The human subject is in one embodiment a healthy human individual, in another embodiment, a human subject who is suffering of or recovering from a disease, in another embodiment, a human subject is an individual who is affected by a physical injury or a psychological or environmental factor. The composition, in one embodiment, is for use in the dietary management of one or more symptoms and conditions associated with an imbalanced, damaged or reduced skeletal muscle energy metabolism, or dietary management of one or more symptoms and conditions associated with a weakened or damaged mitochondrion function in skeletal muscle, wherein the symptom or condition is preferably selected from muscle weakness, muscle pain, low muscle tone, exercise intolerance, lack of muscular endurance and loss of muscle mass.
A composition comprising choline and succinate in the molar ratio about 2:1, or a composition comprising, preferably, essentially consisting of choline, succinate and nicotinamide, or a nicotinamide derivate, in the molar ratio from about 2:1:0.01 to about 2:1:10 for a treating a musculoskeletal disease or musculoskeletal disorder in a subject. The disease or disorder may be selected from muscle-wasting, muscle degenerative disease, myopathies, frailty, pre-frailty, neuromuscular diseases, Duchenne muscular dystrophy, sarcopenia, muscle atrophy and/or cachexia, muscle loss, a muscle function disorder, age-related decline in muscle function, age-related sarcopenia, age-related muscle-wasting, physical fatigue, muscle fatigue, inclusion body myositis, sporadic inclusion body myositis, or from metabolic myopathy, such as acid maltase deficiency (AMD, Pompe disease, glycogenosis type 2, lysosomal storage disease), carnitine deficiency, carnitine palmityl transferase deficiency (CPT deficiency), debrancher enzyme deficiency (Cori or Forbes disease, glycogenosis type 3), lactate dehydrogenase deficiency (glycogenosis type 11), myoadenylate deaminase deficiency, phosphofructokinase deficiency (Tarui disease, glycogenosis type 7), phosphogylcerate kinase deficiency (glycogenosis type 9), phosphogylcerate mutase deficiency (glycogenosis type 10), phosphorylase deficiency (McArdle disease, myophosphorylase deficiency, glycogenosis type 5). The composition is in one embodiment nutritional and formulated as a food supplement, beverage, or food, such as a medical beverage or medical food; in another embodiment, the composition is pharmaceutical.
A method for
(i) the dietary management of a condition or a symptom associated with an imbalanced, damaged or reduced skeletal muscle energy metabolism; or
(i) the dietary mitigation of the risk of occurrence and/or re-occurrence of a symptom or condition associated with an imbalanced, damaged or reduced skeletal muscle energy metabolism
in a human subject, comprising administering to said human at least once a day any composition of the invention.

The symptom or condition is selected from muscle weakness, muscle pain, low muscle tone, exercise intolerance, lack of muscular endurance and loss of muscle mass. The composition may be administered daily in one or more doses for a period of one or more days.
Use of a combination of choline cation, succinate anion (2−) and nicotinamide, or a nicotine derivate in a human subject, for restoring, maintaining and/or enhancing skeletal muscle energy metabolism and/or mitochondrial autophagy; restoring, maintaining and/or enhancing muscular strength and endurance; maintaining and/or enhancing physical performance; treating or reducing the risk of development or re-occurrence of a condition or a symptom associated with an imbalanced, damaged or reduced skeletal muscle energy metabolism and/or autophagy; treating or reducing the risk of or delaying the onset of physical impairment associated with associated with an imbalanced, damaged or reduced skeletal muscle energy metabolism and autophagy,
   in said human subject; preferably, wherein the molar ratio of choline cation:succinate anion (2−):nicotinamide, or nicotinamide derivate, in the combination is from about 2:1:0.01 to about 2:1:10; preferably, wherein the combination comprising choline succinate (2:1) salt. Preferably, the combination is formulated as a nutraceutical or nutritional composition, e.g. the composition is formulated as a beverage or food product, such as a medical beverage or medical food product.
A sport nutritional supplement, food or beverage comprising from around 10 mg to around 5000 mg of a composition essentially consisting of choline and succinate in the molar ratio about 2:1.
A sport nutritional supplement, food or beverage comprising from around 10 mg to around 5000 mg of a composition essentially consisting of choline, succinate and nicotinamide, or a nicotinamide derivate, in the molar ratio from around 2:1:0.01 to around 2:1:10.
A sport nutritional supplement, food or beverage, wherein choline and succinate are present in the form of dicholine succinate salt.
A sport nutritional supplement, food or beverage of any of the above, for maintaining, improving or restoring physical performance, muscular strength and/or muscular endurance in a human subject.

### EXAMPLES

Non-limiting working examples are presented below to illustrative the invention. Embodiments described in the working examples are not in any way limiting the scope of the invention.

### Example 1. Nutritional Compositions of the Invention

Beverage 1. The beverage is prepared by mixing of NAM with DISU in amounts as indicated below and dissolved in 330 ml of water to provide a beverage.

Beverage 1

| Ingredient | Content, per serving |
|---|---|
| NAM | 37 mg |
| DISU | 250 mg |
| D-gluconic acid | qs to pH 6.5 |
| Water | to 330 ml |

The molar ratio choline:succinate:NAM in this beverage is 2:1:0.4).

Beverage 2. The beverage is prepared by mixing of NAM with DISU in amounts as indicated below and dissolved in 330 ml of water.

Beverage 2

| Ingredient | Content, per serving |
|---|---|
| NAM | 188 mg |
| DISU | 250 mg |
| D-gluconic acid | qs to pH 6.5 |
| Water | to 330 ml |

The molar ratio choline:succinate:NAM in this beverage is 2:1:2.

Beverage 3. The beverage is prepared by mixing of NAM with DISU in amounts as indicated below and dissolved in 330 ml of water.

Beverage 3

| Ingredient | Content, per serving |
|---|---|
| NAM | 188 mg |
| DISU | 500 mg |
| D-gluconic acid | qs to pH 6.5 |
| Water | to 330 ml |

The molar ratio choline:succinate:NAM in this beverage is 2:1:1.

Beverage 4. The beverage is prepared by mixing of NAM with DISU in amounts as indicated below and dissolved in 500 ml of water.

Beverage 4

| Ingredient | Content, per serving |
|---|---|
| NAM | 210 mg |
| DISU | 560 mg |
| D-gluconic acid | qs to pH 6.5 |
| Water | to 500 ml |

The molar ratio choline:succinate:NAM in this beverage is 2:1:1.

Beverage 5. The beverage is prepared by mixing of NAM with DISU in amounts as indicated below and dissolved in 330 ml of water.

Beverage 5

| Ingredient | Content, per serving |
|---|---|
| NAM | 375 mg |
| DISU | 100 mg |
| D-gluconic acid | qs to pH 6.5 |
| Water | to 330 ml |

The molar ratio choline:succinate:NAM iin this beverage is about 2:1:10.

Beverage 6. The beverage is prepared by mixing of NAM with DISU in amounts as indicated below and dissolved in 330 ml of water.

Beverage 6

| Ingredient | Content, per serving |
|---|---|
| NAM | 7 mg |
| DISU | 2000 mg |
| Succinic acid | qs to pH 6.5 |
| Water | to 330 ml |

The molar ratio choline:succinate:NAM in this beverage is 2:1:0.01

Beverage 7. The beverage is prepared by mixing of NAM with DISU in amounts as indicated below and dissolved in 330 ml of water to provide a beverage.

Beverage 7

| Ingredient | Content, per serving |
|---|---|
| NAM | 37 mg |
| DISU | 250 mg |
| Citric acid | qs to pH 6.5 |
| Water | to 330 ml |

The molar ratio choline:succinate:NAM in this beverage is 2:1:0.4.

### Example 2. Evaluation of the Effect of Compositions of the Invention on Cellular Energetics and Mitophagy in Primary Cultures of Myocytes

### Materials and Methods.

Primary Skeletal Muscle Cell culture. Primary myotube cells were obtained from postnatal day 0-2 (P0-P2) rat pups which were sacrificed by decapitation. Briefly, the rats were immersed in 70% ethanol and the hind-limbs removed under a dissection microscope, in dissection solution containing Dulbecco's Phosphate Buffered Saline (PBS) with penicillin streptomycin (5000 units/ml) and Amphotericin B (2.5 mg/L). The tissues were then digested in 0.2% collagenase solution at 37ºC for 30-40 minutes. The reaction was stopped by adding 2-3 ml foetal bovine serum (FBS), and the tissue suspension was centrifuged at 2010 rpm (700 g) for 10 minutes. The cell pellet was resuspended in pre-warmed growth medium, Dulbecco's modified Eagle's medium (DMEM), containing 20% FBS, 100 i.u./ml penicillin and 200 i.u./ml streptomycin], and plated on 22 mm coverslips that were pre-coated with 0.2% gelatin for 1 hour at 37° C. Cells were then cultured in a humidified CO2 incubator (5% CO2 in air) at 37° C. After 2 or 3 days, the medium was changed to a low-serum medium (DMEM containing 10% FBS without antibiotics), and was subsequently renewed every 3 or 4 days. All experiments were performed in vitro between days 2 and 11 in culture when spindle-shaped myoblasts had proliferated and aligned themselves for fusion (˜70% confluency).

Effect of compositions of the invention on capacity of primary myocyte (myotubes) mitochondria to produced “energy” molecules ATP, NAD and FAD was assessed in resting cells and activated cells in the cellular model of exercise (myotubes contractions are induced by different caffeine concentrations, as described below).

In particular, the effect of acute application of the compounds (i.e. di-choline succinate (DISU) and nicotinamide (NAM) and a combination thereof) on mitochondrial membrane potential, level of mitochondrial NADH and FAD, and intracellular ATP was studied on resting myotubes using a 24- and 48-hours incubation of the myotubes with the compounds.

Myotubes activated by
1 mM caffeine to induce singe calcium peaks and myotube contractions to simulate ‘physiological’ level of myotube activity resembling the activity of exercise,
5 mM caffeine to stimulate a near maximal [Ca2+]c signal and fast contractions of myotubes, and
10 mM caffeine to cause potentially damaging [Ca2+]c overload, were used as an “exercise model”. The cell cultures are incubated with the compounds to evaluate the effect on the level of production of ATP, NAD, FAD and mitochondrial membrane potential during exercise.

Confocal Microscopy. Confocal images were obtained using an inverted Zeiss 710 uv-vis CLSM equipped with a META detection system and a 40× oil immersion objective. The 488 nm Argon laser line was used to excite fluo-4, MagFluo-4 fluorescence, which were all measured using a bandpass filter of 505-550 nm. Illumination intensity was kept to a minimum (at 0.1-0.2% of laser output) to avoid phototoxicity and the pinhole set to give an optical slice of ˜2 μm.

NADH and its oxidized form NAD act as hydrogen carriers at the site of the electron transport chain during mitochondrial respiration. The fluorescent properties of NADH make it a valuable fluorescent indicator of the mitochondrial metabolic state. NADH autofluorescence was excited at 351 and measured at 375-470 nm.

Tetramethyl rhodamine methyl ester (TMRM) was used to study the changes of the mitochondrial membrane potential (Δψₘ). Cells were loaded with 25 nM TMRM for 40 min until a steady state was achieved. TMRM fluorescence was excited at 543 nm (HeNe laser), and emission was measured at >560 nm. The positively charged lipophilic compound is sequestered into the mitochondria by virtue of their negative potential compared to the cytosol. Therefore, the changes in potential are reflected by changes in fluorescence intensity within the mitochondria which is measured after thresholding out the background signal from the cytosol to give a mean value per pixel within the mitochondrial population of a cell. It is important to emphasize that this measure is completely independent of mitochondrial mass or number in a cell.

Evaluation of the mitochondrial FAD level was done as described above for NADH.

All presented results are obtained from at least 5 coverslips and 2-3 different cell preparations.

### Results.

### 1. Evaluation of the Mitochondrial Membrane Potential (Δψₘ)

Treatment of primary myotube cultures loaded with TMRM with (1) di-choline succinate (DISU, 50 μM), (2) nicotinamide (NAM; 20 μM) or (3) simultaneously with 50 μM DISU and 20 μM NAM (ratio choline:succinate:nicotinamode=2:1:0.4) induced a increase in TMRM fluorescence compared to control. Subsequent application of the 1 μM FCCP induce complete mitochondrial depolarisation and decrease the TMRM signal confirming ability of the compounds to increase mitochondrial membrane potential. The mixture of DISU and NAM (treatment 3) had the highest effect—21±2% (p<0.05; n=67 myotubes), compared to DISU (treatment 1) and NAM (treatment 2).

### 2. Evaluation of the Mitochondrial NADH Pool

NADH is produced in the Krebs cycle inside of mitochondria. It is used as a donor of electrons and substrate for the mitochondrial complex I. NADH is a fluorescent compound and this allows to measure its level in the mitochondria. However, NADH fluorescence cannot be distinguished from the fluorescent signal deriving from NADPH. To separate these two fluorescent signals, mitochondrial activators and inhibitors can be utilized. Accordingly, to measure the resting level of NADH autofluorescence, it was generated the “redox index,” a ratio of the maximally oxidized (response to 1 μM FCCP, a potent uncoupler of mitochondrial oxidative phosphorylation,—mitochondria respiring maximally with minimal level of NADH in mitochondria) and maximally reduced (response to 1 mM NaCN, a potent inducer of decrease in the mitochondrial membrane potential—both respiration and consumption of the NADH in mitochondria is blocked, which leads to the maximal values of NADH—FIG. 1A) signals.

Treatment of myotube cultures with two different blends DISU+NAM (blend 1: 100 μM DISU+40 UM NAM; blend 2: 50 μM DISU+20 μM NAM; the molar ration choline:succinate:NAM=2:1:0.4), or with single compounds of the blends, i.e. choline (100 μM), succinate (50 μM), NAM (50 μM) did not induce significant changes in the redox index (which corresponds to NADH-dependent mitochondrial respiration (FIG. 1B). However, both blends of DISU and NAM, but not the individual components, significantly increased the mitochondrial pool of NADH (measured as a Δ between FCCP and NaCN NADH autofluorescence) (FIG. 1 C).

### 3. Evaluation of the FAD Level

FADH is essential for mitochondrial complex Il and also a biological marker of the functionality of mitochondria.

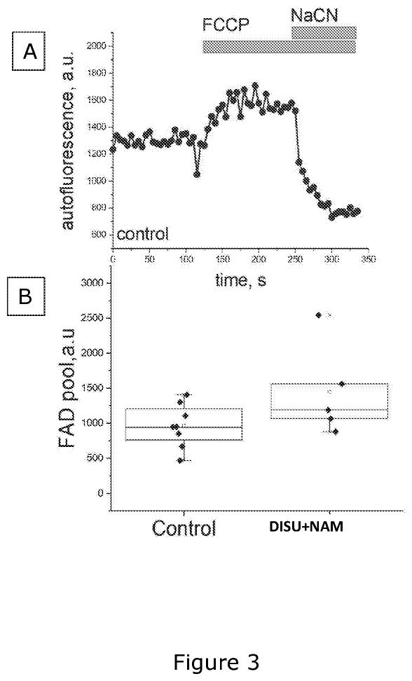

FAD++ is fluorescent and the mitochondrial level of FAD+++ was measured based on evaluation of its autofluorescence (in the same way as described above for NADH) (FIG. 3A). Treatment of myotubes with a blend of DISU and NAM (50 μM DISU+20 μM NAM; the molar ration choline:succinate:NAM=2:1:0.4) resulted in an increase of the mitochondrial pool of FAD++(FIG. 3B).

### 4. Evaluation of the Overall Energy Capacity of the Activated Myotubes

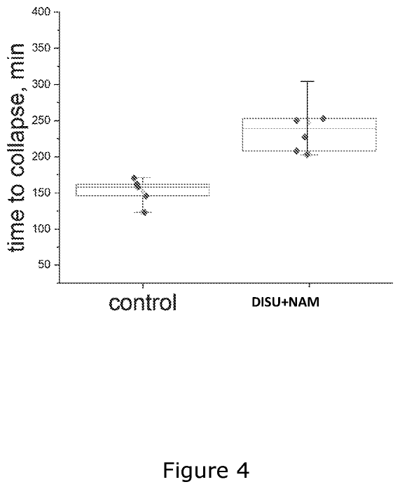

The energy capacity of the cell is defined as the time between cessation of ATP production and the time of energetic collapse due to total ATP depletion and inability to maintain calcium homeostasis. Live-cell imaging of the fluorescent probe MagFura-2 was used to assess the energy capacity primary myotubes. Mg2+ is released from MgATP upon the hydrolysis of ATP, so the measurement of cellular free magnesium ([Mg2+]c) using the Mg2+-sensitive fluorescent probe MagFura-2 can be used as an indication of ATP consumption (Leyssens et al., (1996) J Physiol. 496, 111-128). Application of inhibitors of glycolysis and/or oxidative phosphorylation blocks the ATP production in cells, which eventually leads to ATP depletion and subsequent Mg2+ release and an increase in Mag-Fura fluorescence (FIG. 4). To estimate the overall ATP consumption, inhibitors of glycolysis (iodoacetic acid, 20 μm) and F1F0-ATP synthase (oligomycin, 2 μg/ml) were applied simultaneously. Additionally, the cultures were treated with 1 mM of caffeine to induce calcium dependent myotubes contractions to mimic muscle exercise, which leads to an increased consumption of ATP (FIG. 4).

It is found that treatment of primary myotubes with a blend of 50 μM DISU and 20 μM NAM; the molar ration choline:succinate:NAM=2:1:0.4, significantly increases the time of contraction of myotubes to myotube collapse (from 147±7 to 238±9 min). This surprising finding strongly suggests that simultaneous treatment of myotubes with DISU and NAM increases the myotube energy capacity and thereby enhancing the capability of the muscle cells to sustain contraction for a longer period of time.

### 5. Evaluation of Autophagy

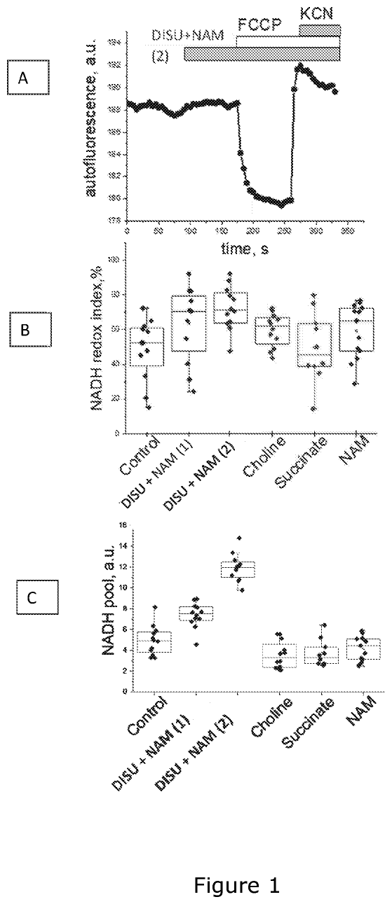

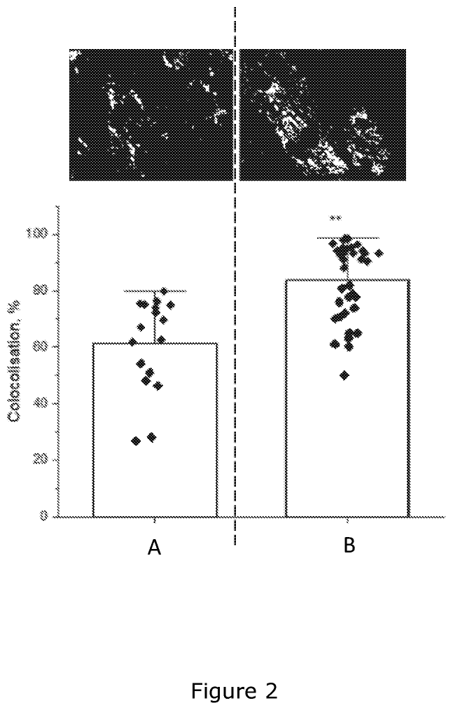

Mitophagy induced by treatments of primary myotubes either with a blend of DISU and NAM (50 μM DISU+20 μM NAM; the molar ration choline:succinate:NAM=2:1:0.4) or single components of the blend (DISU, NAM, choline, succinate (all compounds are used in the molar amounts corresponding to the molar amounts of the compounds in the blend) was evaluated by analysing the level of cellular co-localization of mitochondria (labelled with MitoTracker Green (green color)) and lysosomes (labeled with LysoTracker Red, red color) using confocal microscopy. Both DISU and the blend of DISU and NAM, but not NAM, choline bitartrate or succinate di-sodium salt, were potent to significantly increase autophagy in the treated cultures of primary myotubes. FIG. 2 demonstrates the results of cell treatment with the blend of DISU and NAM vs control (non-treated cells).

### 6. Summary of Evaluation of the Production of ATP, NAD and FAD

Table 1 below summarizes results on the production of ATP, NADH and FAD in resting myocytes in response to treatment with different formulations of choline, succinate and nicotinamide or nicotinic acid. The levels of ATP, NADH and FAD in treated cells were normalized to the levels in control cells

TABLE 1

| Treatment of resting myocytes | ATP | NADH | FAD |
|---|---|---|---|
| Control (no treatment) | − | − | − |
| DISU | ++ | ++ | + |
| Succinate* | + | − | − |
| NAM | + | + | − |
| Nicotinic acid | − | − | − |
| Choline** | + | − | − |
| DISU + NAM | ++++++ | +++++ | +++++ |
| DISU and Nicotinic acid | + | − | − |
| Succinate* and NAM | − | − | − |
| Choline** + Succinate + NAM | +++ | ++ | ++ |

*Sodium succinate (salt);
**Choline bitartrate

In all combined treatments with choline, succinate and NAM/nicotinic acid the molar ratio choline:succinate:NAM/nicotinic acid is 2:1:0.4

The Table 2 below summarizes results on the production of ATP, NADH and FAD in myocytes activated with different concentrations of caffeine in response to treatment with different formulations of choline, succinate and nicotinamide or nicotinic acid. The levels of ATP, NADH and FAD in treated cells were normalized to the levels in control cells.

TABLE 2

| | 1 mM caffein | | | 5 mM caffein | | | 10 mM caffein | | |
|---|---|---|---|---|---|---|---|---|---|
| Treatment | ATP | NADH | FAD | ATP | NADH | FAD | ATP | NADH | FAD |
| Control (no | − | − | − | − | − | − | − | − | − |
| treatment) | | | | | | | | | |
| DISU | ++ | ++ | + | ++ | + | + | + | + | + |
| Succinate* | − | − | − | − | − | − | − | − | − |
| NAM | + | − | − | − | − | − | − | − | − |
| Nicotinic acid | − | − | − | − | − | − | − | − | − |
| Choline** | − | − | − | − | − | − | − | − | − |
| DISU + NAM | +++++ | ++++ | ++++ | ++++ | ++++ | ++++ | +++ | +++ | ++ |
| DISU + Nicotinic acid | + | + | − | − | + | + | + | − | − |
| Succinate* + NAM | + | − | − | − | − | − | − | − | − |
| Choline** + | +++ | ++ | ++ | ++ | + | + | ++ | + | + |
| Succinate* + NAM | | | | | | | | | |

*Sodium succinate (salt);
**Choline bitartrate

In all combined treatments with choline, succinate and NAM/nicotinic acid the molar ratio choline:succinate:NAM/nicotinic acid is 2:1:0.4

The results presented in Table 1 and Table 2 show that
treatment of resting myocytes with the composition consisting of DISU and NAM (molar ratio choline:succinate:NAM=2:1:0.4) results in a significant increase in intracellular levels of both ATP, NADH and FAD compared to the control (p<0.05). The effect of the composition was approximately 1.5-fold greater than sum of the effects of the individual components, DISU and NAM, respectively. This indicates that the compounds of the composition of the invention works in synergy improving production of ATP, NADH and FAD production in resting myocytes;
treatment of activated myocytes with the composition consisting of DISU and NAM (molar ratio choline:succinate:NAM=2:1:0.4) results in a significant increase in intracellular levels of both ATP, NADH and FAD compared to the control (p<0.05) both, in 1 mM, 5 mM and 10 mM caffeine activated cultures. Compared to results of treatment with the single compounds of the composition, the effect is synergetic.

Accordingly, compositions of the invention essentially consisting of choline cation, succinate anion and NAM in the molar ratio about 2:1:04 are effective for the enhancement of production of ATP, NADH and FAD in both resting and activated myocytes in vitro.

According to the invention, compositions where the molar ratio choline:succinate:NAM is within the range of 0.5-3:3-0.5:0.01-10 are effective for enhancing the generation of energy molecules NAD, FAD and ATP in mitochondria and enhancingautophagy in primary cultures of muscle cells. Therefore, the results presented in Tables 1 and 2 are representative and not limiting the scope of the invention to compositions with the molar ratio choline:succinate:NAM of 2:1:0.4.

### Example 3. Study of Skeletal Muscle Function and Endurance Performance in Athletes

### Brief Description of the Study

A randomized, double-blind, placebo-controlled study enrolling at least 36 (18 Elite and 18 Sub-Elite trained endurance male runners. 18 placebo and 18 Beverage 3 are given as a daily oral dose for 4-weeks.

Study details:

| | |
|---|---|
| Allocation: | Randomized |
| Intervention Model: | Parallel Assignment |
| Masking: | Triple (Participant, Investigator, Outcomes |
| | Assessor) |
| Primary Purpose: | Testing of the effect of Beverage 3 |
| Active Comparator: | Dietary Supplement: Beverage 3 (Example 1) |
| | 1 dose of Beverage 3 to be taken daily |
| Placebo | Dietary Supplement: Beverage containing only |
| Comparator: | excipients. |
| | 1 dose of Placebo to be taken daily |

### Eligibility Criteria

### Inclusion Criteria:

Trained elite and sub-elite male runners, age between 18-40 years
Participants will be running >100 km/week
Elite participants—required to have a 3,000 m running personal best time below 9:00 (mm:ss), and/or a VO2max result greater than 65 ml-kg-1·min-1
The sub-elite cohort—required to have a 3,000 m running personal best faster than 10:00 min and/or a VO2max>60 ml-kg-1·min-1

### Exclusion Criteria:

Subjects with diagnosed medical conditions involving thyroid function or other chronic disturbances of metabolic rate
Subjects who are unable to complete the training or testing protocols

### Outcome Measures (Time Frame: 4 Weeks)

### Primary Outcome Measures:

1. Change in plasma levels over time of creatine kinase (CK)
2. Change in race performance time during a 3000 m track race in elite runners

### Secondary Outcome Measures:

1. Change from baseline in aerobic capacity (VO2max)
2. Change from baseline in running economy via indirect calorimetry
3. Change from baseline in lean body mass via Dual-energy X-ray Absorptiometry (DXA)
4. Change from baseline in body fat mass via Dual-energy X-ray Absorptiometry (DXA)
5. Change from baseline in Resting Metabolic Rate (RMR)
6. Change from baseline in maximal muscle strength during 1-repetition maximum leg press
7. Change in iron absorption and hemoglobin mass via determination of carboxyhaemoglobin (percent HbCO) (sub-elite runners only)
8. Change in acylcarnitines levels via metabolomics in plasma
9. change in mitochondrial function via respirometry in muscle biopsies (sub-elite runners only)
10. change in mitochondrial gene expression via RNA-seq in muscle biopsies (sub-elite runners only)
11. change in plasma levels of Urolithin A
12. change in plasma levels of inflammatory marker CRP

### Example 4. Study of Bioenergetics and Muscle Function Improvement in Elderly Skeletal Muscle

### Summary

A randomized, double-blind, single-center, placebo-controlled trial enrolling 50-60 healthy elderly subjects (33 placebo and 33 Beverage 1 administration) who are ≥65 and $90 years of age with evidence of low mitochondrial function. Beverage 1 or Placebo are orally administered for 4 months.

Study Details:

| | |
|---|---|
| Allocation: | Randomized |
| Intervention Model: | Parallel Assignment |
| Masking: | Triple (Participant, Investigator, Outcomes |
| | Assessor) |
| Primary Purpose: | Testing the effect of Beverage 1 |
| Active Comparator: | Dietary Supplement: Beverage 1 (Example 1) |
| | 1 dose of Beverage 1 to be taken daily. |
| Placebo | Dietary Supplement: Placebo beverage (excipients |
| Comparator: | containing beverage) |
| | 1 dose of Placebo beverage to be taken daily |

### Eligibility Criteria:

1. Adult human volunteers of all sexes ≥65 and ≤90 years of age;
2. Capable of walking 6 minute walk distance of <550 meters
3. Have ATP max<1 mM/see (in the hand FDI muscle);

### Exclusion Criteria:

1. Subjects who have significant disease(s) or condition(s) which, in the opinion of the investigator, may put the subject at risk because of their participation in the trial or may influence either the results of the trial or the subject's ability to participate in the trial
2. Hospitalization within 3 months for major atherosclerotic events (defined as combined incidence of myocardial infarction, urgent target-vessel revascularization, coronary bypass surgery and stroke) and for any hospitalization within 2 months.
3. Have any metal implants in the right limbs, including non-MRI compatible metal stents, titanium pins/markers, etc.
4. Have an implanted cardiac pacemaker or other implanted non-MRI compatible cardiac device
5. Chronic, uncontrolled hypertension as judged by the Investigator (i.e., Baseline SBP>150 mm Hg, DBP>90 mm Hg) or a SBP>150 mm Hg or DBP>95 mm Hg at the time of screening or baseline. If the initial BP reading is above these values, the reading may be repeated one time within 20 minutes of the initial reading.
6. Body mass index <18 or >32 kg/m2
7. Severe chronic kidney disease requiring treatment with hemodialysis or peritoneal dialysis.
8. Additional laboratory abnormalities determined as clinically significant by the Investigator.
9. Clinically significant abnormalities on physical examination (as judged by the Investigator)
10. Clinically significant and chronic uncontrolled renal, hepatic, pulmonary, endocrine, neurologic disorders, bone, or gastrointestinal system dysfunction
11. History of seizures or epilepsy
12. History of serious mental illness as judged by the Investigator
13. Oral temperature >37.5° C. at the time of the physical
14. Suspicion, or recent history, of alcohol or substance abuse or tobacco use
15. Subjects who in the opinion of the Investigator have a clinically significant abnormal 12-lead ECG during the screening period. Presence of atrial fibrillation, varying degrees of AV block, existence of a left bundle branch block, or evidence of previous myocardial infarction
16. Subjects who are either unwilling to agree to refrain from using or are found to be using supplementary antioxidant vitamins (e.g., Coenzyme Q10, resveratrol, L-carnitine) from 7 days prior to dosing and throughout the treatment period
17. Subjects who are either unwilling to agree to refrain from using or are found to be using the following dietary restrictions (pomegranate juice, walnuts, pecans, strawberry, raspberry blackberry) from 7 days prior to dosing and throughout the treatment period
18. Are currently enrolled in a clinical trial involving an investigational product or non-approved use of a drug or device or concurrently enrolled in any other type of medical research judged not to be scientifically or medically compatible with this study
19. Have participated, within the last 30 days from a clinical trial involving an investigational product. If the previous investigational product has a long half-life, 3 months or 5 half-lives (whichever is longer) should have passed

### Primary Outcome Measures: (Time Frame:4 Months)

1. Change in 6 minute walking distance (6MWD) at the end of study intervention compared to baseline
2. Percent change from baseline in ATP max (maximal ATP synthesis rate) in hand skeletal muscle (via Magnetic Resonance Spectroscopy)

### Secondary Outcome Measures (Time Frame:4 Months):

1. Percent change from baseline in contraction number during a hand muscle fatigue test
2. Percent change from baseline in ATP max (maximum ATP synthesis rate) in leg skeletal muscle (via MRS)
3. Percent change from baseline in contraction number during a leg muscle fatigue test
4. Change in Short Physical Performance Battery (SPPB) scores at the end of study intervention compared to baseline
5. Change in exercise tolerance compared to baseline (via cycle ergometry)
6. Change in hand grip strength at the end of study intervention compared to baseline
7. Change in leg muscle strength (1-RM and 10-RM) at the end of study intervention compared to baseline
8. Change in muscle size (cross-sectional area of the muscles) at the end of study intervention compared to baseline
9. Change in mitochondrial function on muscle biopsy samples at the end of study intervention compared to baseline (via respirometry)
10. Effect of BEVERAGE 1 on mitochondrial gene and protein expression in muscle tissue
11. Effect of Beverage 1 on plasma acylcarnitines
12. Effect of BEVERAGE 1 on quality of life questionnaire (SF36)
13. Change from baseline in plasma lipid profile
14. Change from baseline in plasma for circulating biomarkers (myostatin, follistatin)

1-39. (canceled)

40. A nutritional supplement comprising:
a composition formulated to maintain, enhance, and/or restore skeletal muscle energy, the composition containing choline and succinate in a molar ratio of choline to succinate of 2:1 in a predetermined dosage amount effective to increase intracellular levels of:
adenosine triphosphate (ATP),
nicotinamide dinucleotide (NADH), and
flavine dinucleotide (FAD),
in skeletal muscle cells of a human subject consuming the predetermined dosage amount at least once daily.

41. The nutritional supplement of claim 40, wherein the predetermined dosage amount is effective in enhancing autophagy in the skeletal muscle cells of the human subject consuming the predetermined dosage amount at least once daily.

42. The nutritional supplement of claim 40, wherein the effective predetermined dosage amount of the composition is within a range of from about 100 mg to about 1000 mg per serving in one or more servings up to a total of about 2000 mg per day.

43. The nutritional supplement of claim 40, wherein the effective dosage amount of the choline and succinate comprises dicholine succinate salt (DiSu).

44. The nutritional supplement of claim 40, further comprising a nicotinamide component selected from nicotinamide, nicotinamide riboside, nicotinamide mononucleotide, and combinations thereof, wherein the molar ratio of the choline to the succinate to the nicotinamide component is 2:1:0.01-10.

45. The nutritional supplement of claim 40, further comprising one or more components selected from creatine, glycine, and arginine.

46. The nutritional supplement of claim 40, wherein the supplement is effective in maintaining, improving, and/or restoring one or more characteristics of healthy skeletal muscle selected from skeletal muscle tone, skeletal muscle strength, and skeletal muscle endurance in the human subject.

47. The nutritional supplement of claim 40, wherein the nutritional supplement is formulated as a sports food or a sports beverage.

48. The nutritional supplement of claim 40, wherein the human subject is an aging human individual or a human individual whose musculoskeletal activity is impaired by a physical injury, a psychological factor, and/or an environmental factor.

49. The nutritional supplement of claim 40, wherein the human subject is an individual whose musculoskeletal activity is affected by a weakened or damaged mitochondrial function in skeletal muscle cells, and the individual has one or more symptoms of imbalanced, damaged, or reduced skeletal muscle energy metabolism, the one or more symptoms selected from muscle weakness, muscle pain, low muscle tone, exercise intolerance, lack of muscular endurance, and loss of muscle mass.

50. The nutritional supplement of claim 40, wherein the nutritional supplement is formulated as a medical food or a medical beverage.

51. A method comprising:
providing for consumption by a human subject, a nutritional supplement formulated to improve physical performance of the human subject, the nutritional supplement comprising:
a composition containing choline and succinate in a molar ratio of choline to succinate of 2:1 in a predetermined dosage amount effective improve in the subject, one or more characteristics of healthy skeletal muscle function selected from skeletal muscle tone, skeletal muscle strength, and/or skeletal muscle endurance.

52. The method of claim 51, wherein the effective predetermined dosage amount of the composition is within a range of from about 100 mg to about 1000 mg per serving in one or more servings up to a total of about 2000 mg per day.

53. The method of claim 51, further comprising a nicotinamide component selected from nicotinamide, nicotinamide riboside, nicotinamide mononucleotide, and combinations thereof, wherein the molar ratio of the choline to the succinate to the nicotinamide component is 2:1:0.01-10.

54. The method of claim 51, wherein the composition further comprises one or more components selected from creatine, glycine, and arginine.

55. A method for dietary improvement of skeletal muscle function in a human subject, the method comprising:
providing for consumption by the human subject, a composition formulated to promote healthy skeletal muscle function in the human subject, the composition containing choline and succinate in a molar ratio of choline to succinate of 2:1 in a predetermined dosage amount effective to reduce symptoms of impaired skeletal muscle energy metabolism in the human subject.

56. The method of claim 55, wherein the effective predetermined dosage amount of the composition is within a range of from about 100 mg to about 1000 mg per serving in one or more servings up to a total of about 2000 mg per day.

57. The method of claim 55, further comprising a nicotinamide component selected from nicotinamide, nicotinamide riboside, nicotinamide mononucleotide, and combinations thereof, wherein the molar ratio of the choline to the succinate to the nicotinamide component is 2:1:0.01-10.

58. The method of claim 55, wherein the composition further comprises one or more components selected from creatine, glycine, and arginine.

59. The method of claim 55, wherein:
the human subject is an aging human individual or a human individual whose musculoskeletal activity is negatively affected by a weakened or damaged mitochondrial function in skeletal muscle cells; and
the predetermined dosage amount is effective in the human subject to improve skeletal muscle cell autophagy and/or reduce one or more symptoms of skeletal muscle dysfunction selected from muscle weakness, muscle pain, low muscle tone, exercise intolerance, and lack of muscular endurance.
